SEQUENCE LISTING (4 sequences)

SEQ ID NO: 1 - DNA, 18 bp, Artificial Sequence
  Primer
ggcaaattcaacggcaca

SEQ ID NO: 2 - DNA, 21 bp, Artificial Sequence
  Primer
gttagtggggtctcgctcctg

SEQ ID NO: 3 - DNA, 22 bp, Artificial Sequence
  Primer
gactgtagaacggtgtcatcaa

SEQ ID NO: 4 - DNA, 22 bp, Artificial Sequence
  Primer
cctgtgaggtaggaatgcgaac

(19) United States
(12) Patent Application Publication
(10) Pub. No.: US 2016/0296722 A1
(43) Pub. Date: Oct. 13, 2016

(54) METHOD OF TREATING DEPRESSION

(71) Applicants: Sung Yeoun0 Hwang, Incheon (KR); Korea Bio Medical Science Institute, Seoul (KR)

(72) Inventors: Sung Yeoun HWANG, Incheon (KR); Hyung Min KIM, Seoul (KR); Hyun-Ja JEONG, Seoul (KR)

(21) Appl. No.: 14/681,912

(22) Filed: Apr. 8, 2015

(51) Int. Cl. A61M 21/02

(52) U.S. Cl. CPC A61M 21/02; A61M 2021/0027

(57) ABSTRACT

A method for treating depression is disclosed. By allowing patients suffering from depression to hear jinyangjo-jangdan (a very slow rhythm) of sanjo (a style of traditional Korean music), the method can be useful in replacing medicinal treatment since the method has an excellent therapeutic effect with a level similar to that when a drug is administered even without administering any drugs, by promoting the activities of monoamine compounds such as serotonin and a brain-derived neurotrophic factor (BDNF) in the brains of patients suffering from depression.

### FIELD OF THE INVENTION

The invention relates to a method for treating depression.

### BACKGROUND OF THE INVENTION

Stress affects our physical and psychological functions, and in the modern world is an inevitable reality. Moreover, chronic stress is associated with headache, chronic fatigue, gastric ulcers, and heart disease as well as depression, and depression affects up to 20% of the world's population. The WHO and many psychiatrists have expressed concerns that depression is likely to become the most common disease in the 21th century. In Korea, many socio-psychological analyses have been conducted on the causes of mental depression, and identified causes include, the rapid disintegration of the family unit, individual isolation, the competition individuals are exposed to in the modern world, and the ‘loose’ structure of the social safety net.

Depression is the result of chemical imbalances in the brain driven by genetic and environmental factors. It is a serious medical condition that affects the body, mood, and thoughts, and those suffering from depression have feelings of sadness and hopelessness that last for considerable periods of time. A combination of medication and psychiatric counseling is mostly commonly used to treat the condition, but recently, several researchers have reported that music therapy can be just as effective as drugs for the treatment of depression, and suggested music be viewed as an alternative means of getting in touch with emotions and developing relationships (Dileo et al., 2009; Gold et al., 2005). Furthermore, a specific music therapy technique has been devised as a means of achieving ‘self-projection and free association’, enabling one to connect with emotional memories and images (Hadley, 2003).

Regarding the classic pathophysiological theory of depression, the monoamine hypothesis holds that depression results from low levels of 5-hydroxytryptamine (5-HT, serotonin) and/or dopamine (DA) in the central nervous system. Depression has also been associated with impaired neurotransmission of 5-HT and DA pathways, although most pharmacologic treatment strategies directly target the enhancement of 5-HT neurotransmission. 5-HT increases levels of brain-derived neurotrophic factor (BDNF), which acts as a potential regulator of memory and synaptic plasticity. In fact, antidepressants increase BDNF expression in the hippocampus. Furthermore, it has been shown that the beneficial effect of BDNF involves the activation of extracellular signal-regulated kinase (ERK). Estradiol also has an antidepressant effect by increasing BDNF expression via the activation of estrogen receptor (ER-β) concluded estradiol was contributed to the anxiolytic effects of music in female mice. Recently, it was reported that neuroinflammatory responses induced by interleukin (IL)-1β, IL-6, and tumor necrosis (TNF)-α produced by microglia in the central nervous system, inhibit the BDNF signal pathway. Accordingly, it appears that deficiencies of monoamine neurotransmitters and BDNF in the central nervous system assume pivotal roles in the pathology and physiology of depression.

Representative types of antidepressants used in such a mechanism include selective serotonin reuptake inhibitors (SSRIs), serotonin-norepinephrine reuptake inhibitors (SNRIs), monoamine oxidase inhibitors (MAOIs), etc. However, since use of such antidepressants often causes side effects such dizziness, drowsiness, blurred vision, rapid heartbeat, mouth dryness, excessive sweating, etc., there is a demand for development of a method for treating depression which can replace medicinal treatment.

### SUMMARY

This invention discloses a method for treating depression, comprising a step of allowing patients suffering from depression to hear jinyangjo-jangdan (a very slow rhythm) of sanjo (a style of traditional Korean music).

In some embodiments of the present invention, the sanjo is daegeum sanjo.

In some embodiments of the present invention, the daegeum sanjo is daegeum sanjo played by Jang-Hyeon WON.

In some embodiments of the present invention, the patients suffering from depression are allowed to hear the jinyangjo-jangdan for 30 minutes or more a day in a period of time of 3 weeks or more.

In some embodiments of the present invention, the treatment of depression is performed by promoting expression of serotonin or dopamine in the brains of the patients suffering from depression.

In some embodiments of the present invention, the treatment of depression is performed by promoting expression of a brain-derived neurotrophic factor (BDNF) in the brains of the patients suffering from depression.

In some embodiments of the present invention, the treatment of depression is performed by promoting phosphorylation of an extracellular signal-regulated kinase (ERK) in the brains of the patients suffering from depression.

In some embodiments of the present invention, the treatment of depression is performed by promoting expression of an estrogen receptor-β (ER-β) in the brains.

In some embodiments of the present invention, the treatment of depression is performed by inhibiting expression of blood inflammatory cytokines

The above objectives and advantages of the present invention will become more readily apparent to those ordinarily skilled in the art after reviewing the following detailed descriptions and accompanying drawings.

### BRIEF DESCRIPTION OF THE DRAWINGS

FIGS. 1A to 1C are diagrams showing immobility times (FIG. 1A), serotonin levels (FIG. 1B), and dopamine levels (FIG. 1C) in the brains of patients in a group treated with jinyangjo-jangdan of daegeum sanjo (JDS music), a fluoxetine-treated group, and an untreated group (blank), all of whom underwent FST tests to cause depression, and a group who underwent no FST test (normal).

FIGS. 2A to 2D are diagrams showing BDNF and ERK ratios in the group treated with JDS music, the fluoxetine-treated group, and the untreated group (blank), all of whom underwent FST tests to cause depression.

FIG. 3 is a diagram showing expression levels of ER-β mRNA in the group treated with jinyangjo-jangdan of daegeum sanjo (JDS music), and the untreated group (blank), all of whom underwent FST tests to cause depression.

FIGS. 4A to 4C are diagrams showing levels of blood inflammatory cytokines in the group treated with JDS music, and the untreated group (blank), all of whom underwent FST tests to cause depression.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for purpose of illustration and description only; they are not intended to be exhaustive or to be limited to the precise form disclosed.

In some embodiments of the present invention, the method for the present invention includes a step of allowing patients suffering from depression to hear jinyangjo-jangdan (a very slow rhythm) of sanjo (a style of traditional Korean music).

Sanjo is a style of instrumental solo within traditional Korean music.

Sanjo includes at least four rhythms including jinyangjo, jungmori, jungjungmori, and jajinmori, and a melody is composed in one of three tonal systems including gyemyeonjo (mi, sol, la, do, and re; tonic: la), pyeongjo (re, mi, sol, la, and do; tonic: sol) and ujo (sol, la, do, re, and mi; tonic: do).

Jinyangjo-jangdan is the slowest rhythm among the sanjo rhythms, and the period of jinyangjo-jangdan is twenty-four quarter notes, and these 24 beats are divided into four parts in sextuple time.

The present inventors have found that expression of serotonin and dopamine in the brains of patients suffering from depression is promoted, and expression of a brain-derived neurotrophic factor (BDNF) is also promoted when the patients suffering from depression are allowed to hear jinyangjo-jangdan of sanjo. Also, they have confirmed that phosphorylation of ERK in the brains of the patients suffering from depression is promoted, expression of ER-β is also promoted, and expression of blood inflammatory cytokines is inhibited. As a result, the present inventors have found that jinyangjo-jangdan of daegeum sanjo has an effect of treating depression.

Jinyangjo-jangdan is the slowest rhythm among the jangdan of pansori and sanjo, and is used in sad and lyrical passages. It is similar to a minor chord in Western music in that it expresses heartbreaking grief and mourning. The effect of treating depression is considered to be derived from the fact that the jinyangjo-jangdan provides a catharsis by means of such sad and mourning rhythms.

Sanjo is classified into gayageum sanjo, geomungo sanjo, daegeum sanjo, piri sanjo, haegeum sanjo, ajaeng sanjo, and danso sanjo according to the musical instrument played by a musician. The sanjo according to one exemplary embodiment of the present invention may include gayageum sanjo, geomungo sanjo, daegeum sanjo, piri sanjo, haegeum sanjo, ajaeng sanjo, danso sanjo, etc., and preferably daegeum sanjo. This is because the sounds of the daegeum are interpreted as sighs (grievous sounds).

Daegeum (a traditional Korean transverse flute, Korean bamboo flute) has one blowing hole, six finger holes, and an extra hole covered with a thin membrane called ‘Cheong’ (located between the blowing hole and finger holes). Cheong is a white resonant membrane cut from a reed and makes this instrument produce a distinctive refined, calm sound.

Sanjo is traditional music which is re-created and changed into a new form of music by adding its own melodies to traditional tunes or changing the melodies. Therefore, various schools of sanjo have been formed and handed down.

Specific examples of the daegeum sanjo existing in Korea include eight schools of sanjo played by Beak-Cheon KANG, Dong-Jin KIM, Jong-Gi PARK, Yong-Seon SEO, Saeng-Gang LEE, Jang-Hyeon WON, Beom-Soo HAN, and Ju-Hwan HAN. In the case of the sanjo according to one exemplary embodiment of the present invention, the daegeum sanjo is not limited to a certain school, and may belong to any of the exemplified schools of sanjo.

The present inventors have found that jinyangjo-jangdan of daegeum sanjo played by Jang-Hyeon WON has a therapeutic effect when the patients suffering from depression are allowed to hear the jinyangjo-jangdan of daegeum sanjo played by Jang-Hyeon WON. However, the other schools of sanjo are essentially identical to the daegeum sanjo played by Jang-Hyeon WON in that various schools of sanjo have common modes (i.e., ujo, pyeongjo, and gyemyeonjo) to form sanjo, and the jangdan also include jinyangjo, jungmori, jungjungmori, and jajinmori. Therefore, the other schools of sanjo are also considered to have an effect of treating depression like the daegeum sanjo played by Jang-Hyeon WON.

The term “patient” refers to an animal, preferably a mammal, and most preferably a human, who is the object of treatment, observation or experiment. The mammal may be selected from the group consisting of mice, rats, hamsters, gerbils, rabbits, guinea pigs, dogs, cats, sheep, goats, cows, horses, giraffes, platypuses, primates, such as monkeys, chimpanzees, and apes. In some embodiments, the subject is a human.

BDNF, ERK, ER-β, blood inflammatory cytokines may be independently derived from the patient. In this case, these proteins may have amino acid sequences known in the related art. Blood inflammatory cytokines may be at least one protein selected from the group consisting of TNF-α, IL-1β, IL-6.

A time when the patients suffering from depression are allowed to hear jinyangjo-jangdan of sanjo is not particularly limited, and may be properly selected according to a treatment purpose, a condition of a patient, a background, etc. For example, the patients may be allowed to hear the jinyangjo-jangdan of sanjo for 5 minutes to 60 minutes a day, for example, 5 minutes to 60 minutes, 10 minutes to 60 minutes, 20 minutes to 60 minutes, and 30 minutes to 60 minutes a day. In an aspect of improvement of depression, the patients may preferably be allowed to hear the jinyangjo-jangdan of sanjo for 30 minutes or more a day.

Such music treatment may be performed for a period of time of one day or several years, for example, one day to a year, one day to 6 months, 10 days to 6 months, 15 days to 60 days, 21 days to 180 days, 21 days to 365 days, according to the conditions of a patient. Preferably, the music treatment may be performed for a period of time of 3 weeks or more.

### EXAMPLE

Materials and Methods

Animals

Male ICR mice (3 weeks old) weighing 10-12 g were purchased from the Dae-Han Experimental Animal Center (Daejeon, Republic of Korea), and subsequently maintained at the College of Korean Medicine, Kyung Hee University. Animals were housed 5-10% in as laminar air-flow room maintained at a temperature of 22±1° C. and a relative humidity of 55±10% under a 12:12 L/D cycle light (on at 7:00 h) throughout the study. Food and water were provided ad libitum. All manipulations were carried out between 9:00 and 16:00 h and no animal was used more than once. The study protocol was approved beforehand by the institutional animal care use committee of Kyung Hee University (KHUASP(SE)-10-032).

Music treatment

ICR mice were divided into five groups according to FST results: the untreated control group (without JDS music), the fluoxetine group (the positive control), and JDS music 10 min, 20 min, or 30 min groups. Background sound levels in the special isolation booth constructed on the non-treated control and fluoxetine groups were 10˜40 dB and in the three music treatment groups was 70 dB. Mice were exposed to JDS music daily for 3 weeks. All experiments were carried out in a quite environment other sounds. In this study, JDS of the Jang-Hyun Won genre was used, and was played by Prof Hyung-Min Kim, who is a disciple of Won. The music was recorded and copied using a MP3 player to ensure precisely the same music was played for mice in all treatment groups.

FST

After measuring immobility times, 10 mice were allocated to each of the five study groups in an immobility time-matched manner. Fluoxetine was dissolved in distilled water (D.W). Fluoxetine (10 mg/kg, a classical antidepressant) was orally administered to mice once daily for 3 weeks using an atraumatic feeding needle. The FST was performed as the end of the 3 weeks administration period. During the 6 min FST, immobility times were measured as described by Porsolt et al. (1977). The FST apparatus consisted of two Plexiglas cylinders (height: 25 cm, diameter: 10 cm) placed side by side in a Makrolon cage filled with water at 23-25° C. Two mice were tested simultaneously for 6 min period inside the vertical Plexiglas cylinders; a nontransparent screen was placed between the two cylinders to prevent mice from seeing each other during the test. Total duration of immobility, during the 4 min period from 2 to 6 min was recorded. A mouse was considered immobile when it ceased struggling and remained floating motionless on the water, making only movements necessary to keep its head above water.

5-HT and DA Assay

5-HT levels in brain were measured a 5-hydroxytryptamine assay kit (MyBiosource, San Diego, Calif., USA), and levels of DA in brain were measured using the DA ELISA kit (Genway Biotech Inc., San Diego, Calif., USA).

Isolation of Tissue Protein

Immediately after the FST, mice were sacrificed, brains were rapidly removed, and frozen in liquid nitrogen. Samples were dissected and homogenized in ice-cold buffer supplemented with 0.2 mM DTT, 0.5 mM sodium vanadate, and protease inhibitors. NaCl was then added to a final concentration of 0.45 M, and homogenates were centrifuged at 15,000×g for 30 min. Supernatants were collected and stored at −70° C. Samples were subjected to Western blot analysis for ERK, phosphorylated-ERK (_{P}ERK), BDNF, and GAPDH.

Western Blot Analysis

Supernatant samples obtained as described above were heated at 95° C. for 5min and briefly cooled on ice. After centrifugation, 50 μg aliquots were resolved by 12% SDS-polyacrylamide gel electrophoresis. Proteins were then transferred to nitrocellulose membranes, which were blocked for 2 h with phosphate-buffered saline (PBS) containing Tween-20 (PBST) containing 6% bovine serum albumin. Membranes were incubated overnight at 4° C. with primary antibodies diluted 1:500 with PBST, and washed nine times for 10 min with PBST. For protein detection, blots were incubated with secondary antibodies conjugated peroxidase (1:5000) for 2 h. Finally, protein bands were visualized using an enhanced chemiluminescence kit (GE Healthcare, Piscataway, N.J., USA) according to the manufacturer's instructions.

Enzyme-Linked Immunosorbent Assay (ELISA)

Cytokine levels in serum were measured by ELISA. The ELISA was performed by coating 96-well plates with 1 μg/well of capture antibodies for IL-1β, IL-6, and TNF-α. Before the subsequent steps in the assay, the coated plates were washed twice with PBST. All reagents and coated wells used in this assay were incubated for 2 h at room temperature. The standard curve was generated from known concentrations of cytokine, as provided by the manufacturer. After exposure to the medium, the assay plates were exposed sequentially to each of the biotin-conjugated secondary antibodies, and avidin-peroxidase, and 2′-azino-bis (3-ethylbenzithiazoline-6-sulfonic acid) substrate solution containing 30% H2O2. The plates were read 405 nm. Appropriate specificity controls were included, and all samples were run according to the total protein. The protein was estimated using the bicinchoninic acid method.

Quantitative Real Time-PCR Analysis

Quantitative real time-PCR was performed using a SYBR Green master mix and mRNA levels were analyzed using an ABI StepOne RT-PCR System (Applied Biosystems, Foster City, Calif., USA). Primer sequences for the reference gene (GAPDH) and genes of interest were as follows: GAPDH (5′ GGC AAA TTC AAC GGC ACA 3′ (SEQ ID NO:1) ; 5′ GTT AGT GGG GTC TCG CTC CTG 3′ (SEQ ID NO:2)); and ER-β (5′ GAC TGT AGA ACG GTG TCA TCA A 3′ (SEQ ID NO:3); 5′ CCT GTG AGG TAG GAA TGC GAA C 3′ (SEQ ID NO:4)). The quantitative PCR amplification protocol was as follows: 2 min at 50° C. and 10 min 95° C., followed by 40 cycles of 15 s at 95° C. and 1 min at 60° C., with data collection during the last 30 s.

Statistical Analysis

Results are presented as means±SEMs. The independent t-test and ANOVA with Tukey's post hoc test were used to determine statistical significance. SPSS statistical software (SPSS Inc., Chicago, Ill., USA) was used throughout, and statistical significance was accepted for p values<0.05.

Results

Effect of JDS Music on Immobility

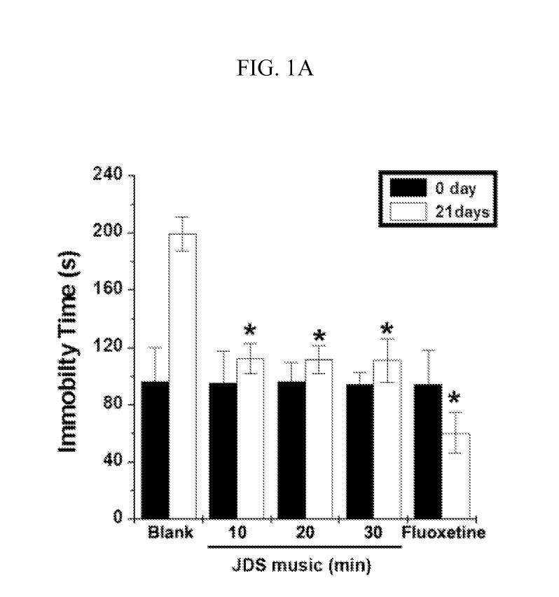

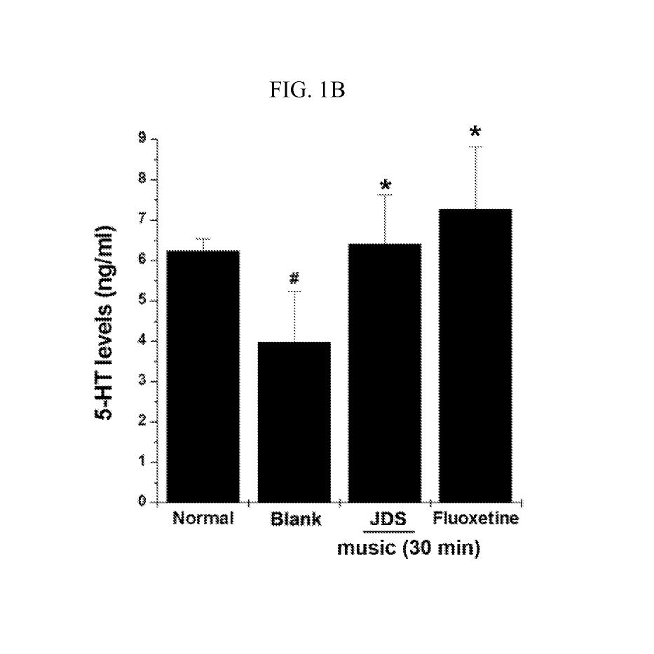

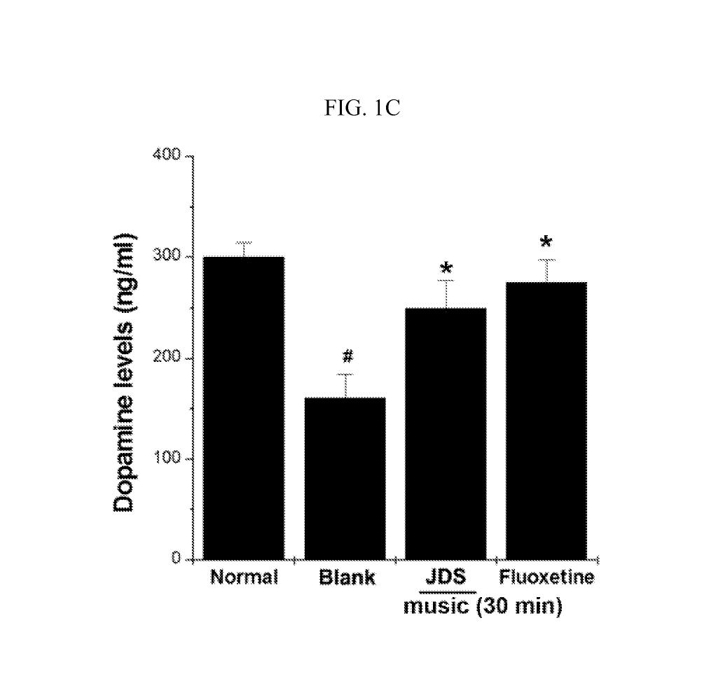

Immobility times were determined 1 h after fluoxetine or JDS music treatment (10, 20, and 30 min). Mean baseline immobility times (0 day) were similar in all five study groups {fluoxetine group (94.2±23.9 s), JDS music groups (95.6±21.9 s for 10 min, 96.2±13.4 s for 20 min, and 94.0±8.4 s for 30 min), and untreated control group (96.0±24.1 s)}. However, after the 3-week treatment period mean immobility times in the fluoxetine (60.4±14.3 s) and JDS music groups (112.4±10.9 s for 10 min, 111.6±9.8 s, for 20 min, and 111.0±15.1 s for 30 min) were significantly lower than in the untreated control group (199.3±11.8 s) (FIG. 1A, p<0.05).

Effect of JDS Music on 5-HT and DA Levels

Selective serotonin reuptake inhibitors have an antidepressant effect by blocking serotonin reuptake at brain synapses. DA is also involved in the pathophysiology and treatment of depression and research studies on DA have led to the developments of a new class of antidepressants. Thus, we analyzed 5-HT and DA levels mouse brains after FST. We found 5-HT and DA levels were significantly lower in untreated control group than in non-FST tested normal control (FIG. 1B and c, p <0.05). However, 5-HT and DA levels in the JDS music and fluoxetine groups were significantly higher than in the untreated control group (FIG. 1B and c, p<0.05).

Effect of JDS Music on Expression of BDNF and Phosphorylation of ERK

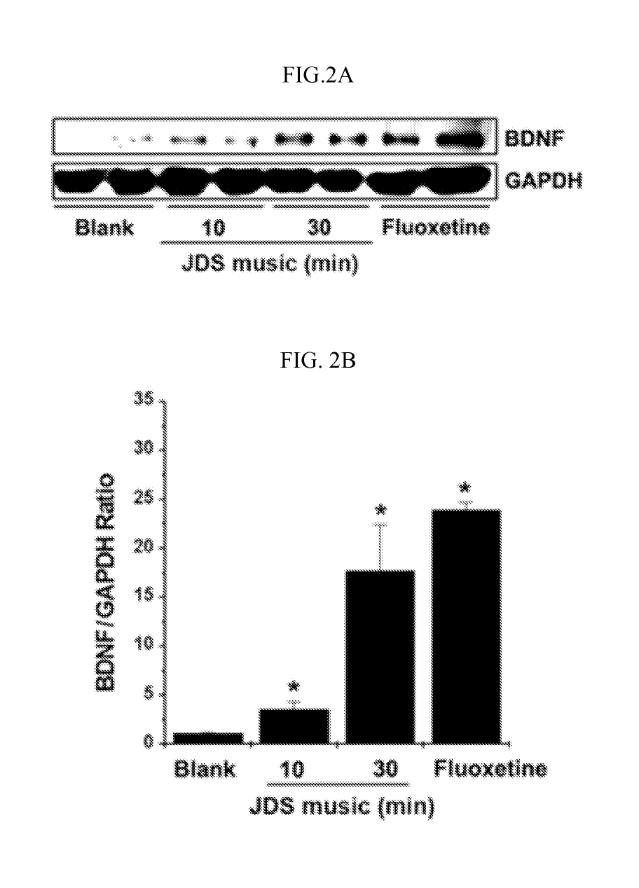

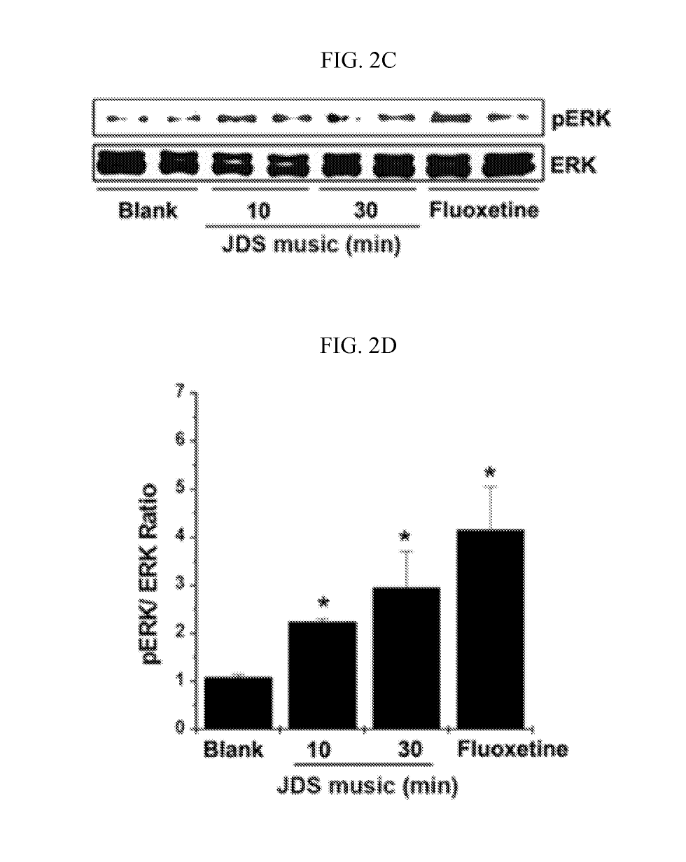

BDNF is a regulator of memory and synaptic plasticity and induces neurogenesis in the brain. As shown in FIGS. 2A and B, JDS music and fluoxetine significantly increased BDNF levels as compared with the untreated control group. The ERK pathway is a key pathway downstream of BDNF and plays an important role in the actions of antidepressants. Therefore, we examined whether the ERK pathway was involved in the antidepressant effect of JDS music. As shown in FIGS. 2C and D, mice exposed to JDS music showed significant increases in pERK (p<0.05), whereas total ERK protein levels were not altered. Similarly, fluoxetine also significantly elevated pERK levels (FIG. 2C and d, p<0.05).

Effect of JDS Music on Expression of ER-β

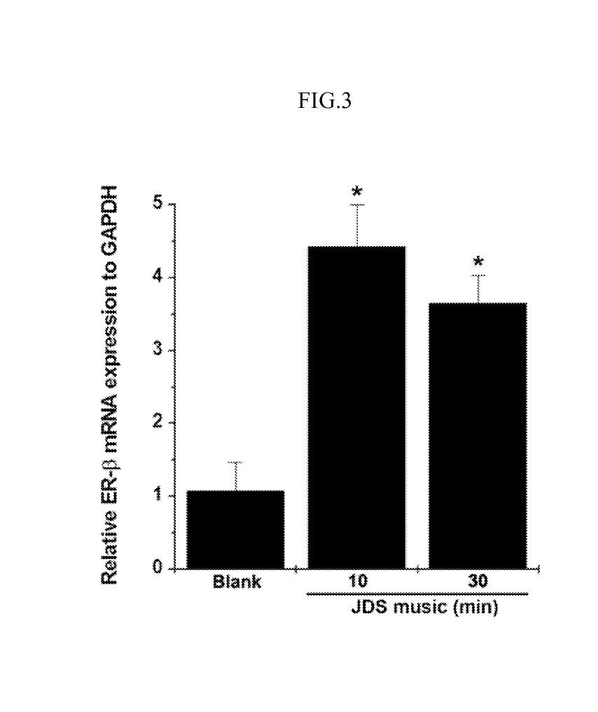

We next investigated whether the estradiol pathway was associated with the antidepressant effect of JDS music. Quantitative real time-PCR showed that JDS music significantly increased the expression of ER-β mRNA (FIG. 3, p<0.05).

Effect of JDS on Levels of Inflammatory Cytokines

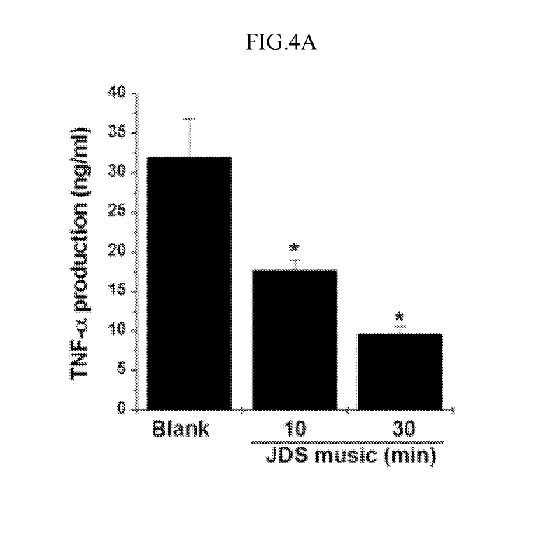

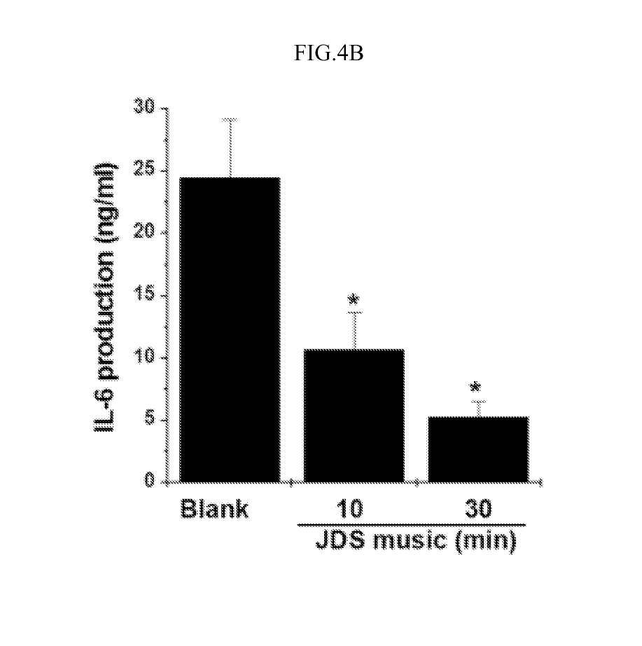

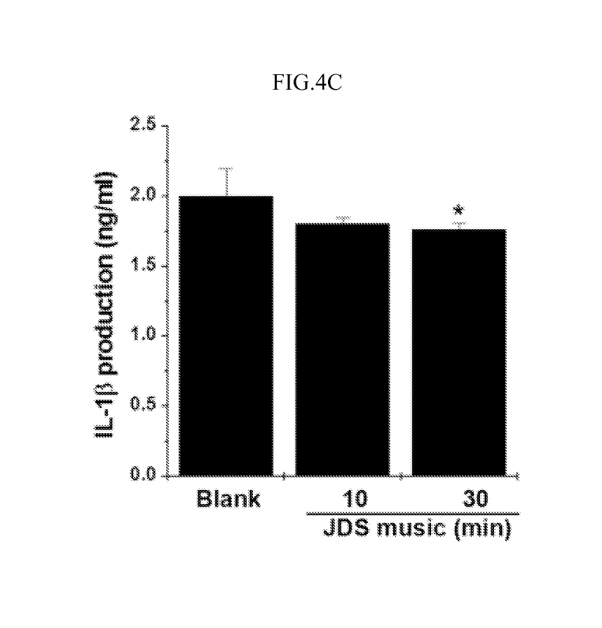

The severity of depression has been showed to be associated with inflammatory cytokine levels. Thus, we analyzed serum protein levels of TNF-α, IL-1β, and IL-6 by ELISA. JDS music (10 and 30 min) significantly reduced serum TNF-α and IL-6 levels versus untreated controls (FIG. 4A and b, p<0.05). However, serum IL-1β levels were only significantly reduced in the JDS music 30 min group (FIG. 4C, p<0.05).

Discussion

In the present invention, JDS music increased the brain expressions of BDNF and ER-β and reduced the levels of inflammatory cytokines, which suggests the antidepressant effect of JDS is associated with activation of BDNF pathways.

In conclusion, this results show JDS music exhibits antidepressant-like effects as determined by the FST, and that these effects appear to be related to modulations of serotonergic system and BDNF signaling pathway in brain.

What is claimed is:

1. A method for treating depression, the method comprising:
allowing a patient suffering from depression to hear jinyangjo-jangdan of sanjo.

2. The method according to claim 1, wherein the sanjo is daegeum sanjo.

3. The method according to claim 2, wherein the daegeum sanjo is daegeum sanjo played by Jang-Hyeon WON.

4. The method according to claim 1, wherein the patients suffering from depression are allowed to hear the jinyangjo-jangdan for 30 minutes or more a day in a period of time of 3 weeks or more.

5. The method according to claim 1, wherein the treatment of depression is performed by promoting expression of serotonin or dopamine in the brains of the patients suffering from depression.

6. The method according to claim 1, wherein the treatment of depression is performed by promoting expression of a brain-derived neurotrophic factor (BDNF) in the brains of the patients suffering from depression.

7. The method according to claim 1, wherein the treatment of depression is performed by promoting phosphorylation of an extracellular signal-regulated kinase (ERK) in the brains of the patients suffering from depression.

8. The method according to claim 1, wherein the treatment of depression is performed by promoting expression of an estrogen receptor-β (ER-β) in the brains.

9. The method according to claim 1, wherein the treatment of depression is performed by inhibiting expression of blood inflammatory cytokines.
